SEQUENCE LISTING (2 sequences)

SEQ ID NO: 1 - DNA, 20 bp, synthetic construct
  source 1..20: mol_type = other DNA; note = nucleotide sequence for a 16S rRNA universal primer, 27F
agagtttgatcctggctcag

SEQ ID NO: 2 - DNA, 19 bp, synthetic construct
  source 1..19: mol_type = other DNA; note = nucleotide sequence for a 16S rRNA universal primer, 1492R
ggttaccttgttacgactt

(19) United States
(12) Patent Application Publication
(10) Pub. No.: US 2025/0312389 A1
(43) Pub. Date: Oct. 9, 2025

(54) PARABACTEROIDES DISTASONIS ISOLATED FROM RUMEN OF CALF AND USE THEREOF

(71) Applicant: Institute of Feed Research of CAAS, Beijing (CN)

(72) Inventors: Yan TU, Beijing (CN); Xiaoran FENG, Beijing (CN); Shengyang XU, Beijing (CN); Chuntao YANG, Beijing (CN); Junnan MA, Beijing (CN)

(21) Appl. No.: 19/079,052

(22) Filed: Mar. 13, 2025

(30) Foreign Application Priority Data
Apr. 8, 2024 (CN) 202410416894.6

(51) Int. Cl. A61K 35/741; A61K 35/00; A61P 1/14; C12N 1/20; C12R 1/01

(52) U.S. Cl. CPC A61K 35/741; A61P 1/14; C12N 1/205; A61K 2035/115; C12R 2001/01

(57) ABSTRACT

A Parabacteroides distasonis strain isolated from rumen of calf and use thereof are provided, relating to the technical field of microorganisms. Provided is a Parabacteroides distasonis strain that simultaneously increases proportions of propanoic acid and butyric acid in a rumen of a ruminant. The strain shows desirable passage stability, safety and resistance, is beneficial to improving an internal environment of the rumen, and maintaining a microecological balance in the rumen; the strain is also beneficial to promoting fermentation in the rumen, promoting nutrient digestion of animal, and enhancing animal immunity. The Parabacteroides distasonis strain was isolated from the rumen of a Holstein cow and can be used as a probiotic additive to feed animals, thereby exhibiting great prospects in the expansion of microbial inoculants for ruminants and the feed industry.

### CROSS REFERENCE TO RELATED APPLICATION

This patent application claims the benefit and priority of Chinese Patent Application No. 2024104168946, entitled “PARABACTEROIDES DISTASONIS Isolated from Rumen of Calf and Use Thereof” filed with the China National Intellectual Property Administration on Apr. 8, 2024, the disclosure of which is incorporated by reference herein in its entirety as part of the present application.

### REFERENCE TO SEQUENCE LISTING

A computer readable XML file entitled “GWP20241207934-Sequence Listing”, that was created on Mar. 6, 2025, with a file size of about 3,325 bytes, contains the sequence listing for this application, has been filed with this application, and is hereby incorporated by reference in its entirety.

### TECHNICAL FIELD

A Parabacteroides distasonis strain isolated from rumen of calf and use thereof are provided, relating to the technical field of microorganisms.

### BACKGROUND

There are a large number of microorganisms in the rumen of ruminants, including bacteria, archaea, anaerobic fungi, protozoa, and bacteriophages. The presence of such microorganisms enables ruminants to better utilize plant-based feed to meet their energy demands, thereby exerting an excellent livestock production performance. The rumen microflora establishes closely related to the host, and by regulating the rumen microflora, rumen fermentation, rumen health level, and livestock production performance can be improved, moreover, the energy utilization. At the same time, the addition of microbial inoculants has gradually received attention in the context of comprehensive antibiotic replacement.

Volatile fatty acids (VFAs) are a main energy source for ruminants, of which propanoic acid, as a precursor for use in glucose synthesis, when present at a high content, it means that ruminants can get more energy for growth, most of butyric acid is converted into O-hydroxybutyrate during absorption in the rumen, which provides an energy source for animal muscles and has a function of repairing the intestinal barrier.

Parabacteroides distasonis is a Gram-negative anaerobe mainly exists in the digestive tract of human beings and animals, and can produce VFAs such as acetic acid and propanoic acid, as well as a large amount of succinic acid. In previous reports, Parabacteroides distasonis is considered a human probiotic as well as one of the 18 core bacterial species in the human intestinal flora. Parabacteroides distasonis has important physiological effects on the human body, which has certain probiotic effects when applied on mice and aquatic animals.

In the prior art, there is still a lack of research on the beneficial effects of Parabacteroides distasonis on ruminants, especially the effects of Parabacteroides distasonis derived from rumen on rumen fermentation parameters and internal environment. It has become a technical problem that needs to be solved in this field to use the Parabacteroides distasonis as an additive to affect rumen fermentation in the ruminants.

### SUMMARY

The present disclosure is proposed to solve the above-mentioned problems in the prior art.

In a first aspect, the present disclosure provides a Parabacteroides distasonis strain, which is designated as Parabacteroides distasonis F4 with a deposit number of CGMCC No. 40798.

In the present disclosure, the Parabacteroides distasonis F4 was isolated from the rumen contents of Holstein calves. The strain was deposited in the China General Microbiological Culture Collection Center (CGMCC) on Dec. 27, 2023, with a taxonomic designation of Parabacteroides distasonis and a deposit number of CGMCC No. 40798; and the CGMCC is located at Institute of Microbiology, Chinese Academy of Sciences, Building No. 3, No. 1 West Beichen Road, Chaoyang District, Beijing 100101, China.

The microbiological characteristics of this strain are: a Gram-negative bacterium, short rod-shaped, non-spore-forming, strictly anaerobic; its colony surface is smooth, and colony edge forms a white or transparent bacterial ring, which is slightly protruding. The strain has a wide range of bile acid conversion functions and can hydrolyze a variety of conjugated bile acids and convert them into a variety of secondary bile acids (lithocholic acid, ursodeoxycholic acid and the like), while also producing a large amount of succinic acid.

The isolation and purification method of the strain includes: collecting fresh rumen fluid samples and diluting them with sterile PBS by 10⁴, 10⁵, 10⁶, and 10⁷ times for plate coating, culturing using fastidious anaerobe agar (FAA) as a medium at 37° C. for 3 d to 5 d under an anaerobic environment, purifying the strain three times by a streak plate method, then conducting microbial mass spectrometry identification or 16S rRNA identification.

In a second aspect, the present disclosure provides a microbial inoculant, which includes the Parabacteroides distasonis F4 or a fermentation broth thereof.

In some embodiments, the fermentation broth is prepared by fermenting the Parabacteroides distasonis F4 in a fastidious anaerobe broth (FAB) medium.

In some embodiments, a preparation method of the fermentation broth includes: inoculating the Parabacteroides distasonis F4 activated to a logarithmic phase into the fermentation medium (FAB) at an inoculation amount of 1% to 5%, and culturing for 12 h to 48 h at a growth temperature of 37° C. to 39° C. and a growth pH value of 6 to 7.

In some embodiments, the fermentation medium (FAB) includes the following components:
mixed peptone 15 g/L, yeast extract 10 g/L, sodium thiglycolate 0.5 g/L, sodium chloride 2.5 g/L, agar 0.75 g/L, cysteine hydrochloride 0.5 g/L, hemin chloride 0.005 g/L, sodium bicarbonate 0.4 g/L, resazurin 0.001 g/L, vitamin K 0.0005 g/L, added distilled water to 1,000 mL, pH=7.0±0.1.

In a specific implementation, the fermentation medium (FAB) is subjected to autoclaving at 121° C. for 15 min before use.

In some embodiments, the microbial inoculant is a freeze-dried powder of the fermentation broth of the Parabacteroides distasonis F4.

In some embodiments, the microbial inoculant contains not less than (1.0×10⁸-1.0×10⁹) cfu/mL or (1.0×10⁸-1.0×10⁹) cfu/g of the live Parabacteroides distasonis F4.

In some embodiments, the freeze-dried powder of the fermentation broth further includes a cryoprotectant; and the cryoprotectant is at least one selected from the group consisting of a skimmed milk powder, dextrin, lactose, and sucrose.

In a third aspect, the present disclosure provides a product, including the Parabacteroides distasonis F4 or the microbial inoculant; where the product is any one selected from the group consisting of a feed or a feed additive.

In a fourth aspect, the present disclosure provides use of the Parabacteroides distasonis F4 or the microbial inoculant in preparation of a product, where the product is any one selected from the group consisting of a feed or a feed additive.

In some embodiments, the product is used in at least one of the following aspects:
(1) increasing proportions of propanoic acid and butyric acid in a rumen of a ruminant;
(2) improving a fermentation pattern of the rumen of the ruminant;
(3) maintaining a microecological balance in the rumen of the ruminant;
(4) promoting digestion of nutrients by the ruminant; and
(5) enhancing an immunity of the ruminant.

In a fifth aspect, the present disclosure provides use of the Parabacteroides distasonis F4, the microbial inoculant, or the product in increasing proportions of propanoic acid and butyric acid in a rumen of a ruminant.

It is known in the art that the proportions of propanoic acid and butyric acid in the rumen of the ruminant cannot directly indicate a specific disease, such that the use does not involve a method for diagnosing or treating a disease.

In a sixth aspect, the present disclosure provides a method for increasing propanoic acid and butyric acid in a rumen of a ruminant, including: adding the Parabacteroides distasonis F4 or a fermentation broth thereof or a freeze-dried powder of the fermentation broth thereof into a feed and feeding the ruminant.

In some embodiments, the Parabacteroides distasonis F4 is added in an amount of more than 1×10⁸ colony-forming unit (CFU) per kg of the feed.

In some embodiments, the ruminant is a Holstein cow.

Compared with the prior art, the present disclosure has the following beneficial effects:

Provided is a Parabacteroides distasonis strain that simultaneously increases proportions of propanoic acid and butyric acid in a rumen of a ruminant. The strain shows desirable passage stability, safety and resistance, is beneficial to improving an internal environment of the rumen, and maintaining a microecological balance in the rumen; the strain is also beneficial to promoting fermentation in the rumen, promoting nutrient digestion of animal, and enhancing animal immunity. The Parabacteroides distasonis strain was isolated from the rumen of a Holstein cow and can be used as a probiotic additive to feed the animals, thereby exhibiting great prospects in the expansion of microbial inoculants for ruminants and the feed industry.

### BRIEF DESCRIPTION OF THE DRAWINGS

FIG. 1 is an image showing a morphology in a solid medium of the Parabacteroides distasonis F4 strain isolated and screened from the rumen of calf in the present disclosure;

FIG. 2 is a 1,000-fold enlarged view of a morphology of the Parabacteroides distasonis F4 strain isolated and screened from the rumen of calf under an oil immersion objective;

FIG. 3 shows a growth curve of the Parabacteroides distasonis F4 strain isolated and screened from the rumen of calf in the present disclosure;

FIG. 4 is a diagram showing acid and alkali resistance of the Parabacteroides distasonis F4 strain isolated and screened from the rumen of calf in the present disclosure;

FIG. 5 is a diagram showing bile salt resistance of the Parabacteroides distasonis F4 strain isolated and screened from the rumen of calf in the present disclosure;

FIG. 6 is a diagram showing artificial gastrointestinal fluid resistance of the Parabacteroides distasonis F4 strain isolated and screened from the rumen of calf in the present disclosure; and

FIG. 7 shows LefSe analysis results of Example 6 in the present disclosure.

### DETAILED DESCRIPTION OF THE EMBODIMENTS

In order to make the objectives, technical solutions, and advantages of the present disclosure clearer, the technical solutions in the present disclosure are described clearly and completely below. Apparently, the described examples are some rather than all of the examples of the present disclosure. All other examples obtained by those of ordinary skill in the art based on the examples of the present disclosure without creative efforts shall fall within the protection scope of the present disclosure.

The techniques or conditions not specified in the examples are all conventional methods or the techniques or conditions described in the literatures in the field, or those according to the product instructions. Reagents or instruments not specified with manufacturers are all conventional products that can be purchased through regular ways.

### Example 1

Isolation and purification of Parabacteroides distasonis F4:

Rumen fluid samples were collected from 6 Holstein calves, where the calves were healthy young with desirable growth performance. The samples were filtered through four layers of gauze and placed in a thermos cup preheated at 39° C. and brought back to the laboratory.

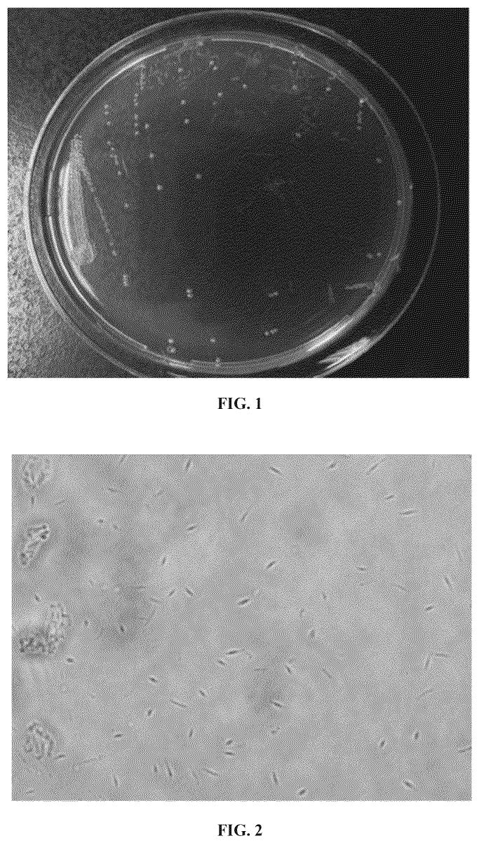

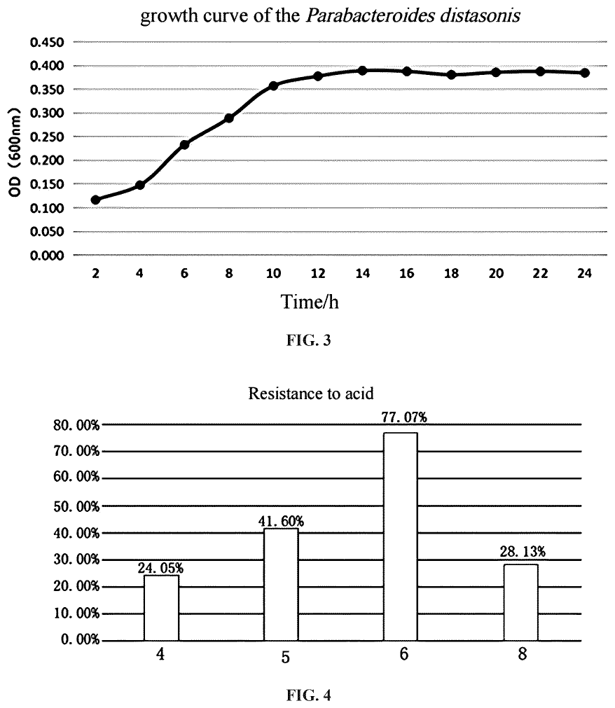

1 mL of the above sample was taken and added into a test tube containing 9 mL of sterile PBS, mixed together with a vortex mixer, and gradient dilution was conducted under sterile conditions. 0.1 mL of dilution solution was taken from each of the three dilutions of sample bacterial suspension at 10⁻³, 10⁻⁴, and 10⁻⁵, coated on FAA solid plates, and cultured anaerobically at 37° C. for 96 h to 144 h, respectively. Some single colonies on the medium plate were selected for four-zone streaking, placed at 37° C. to allow anaerobic culture for 24 h to 48 h, and streaking was repeated 2 to 3 times to obtain purified strains. The morphologies of the strains on a solid plate were shown in FIG. 1, and a 1,000-fold enlarged view of the strains under an oil immersion objective was shown in FIG. 2.

### Example 2

Identification of Parabacteroides distasonis F4:

DNA was extracted from the isolated colonies using a kit purchased from TIANGEN Biotech (Beijing) Co., Ltd. 16S rRNA universal primers (27F: 5′-AGAGTTTGATCCTGGCTCAG-3′ (SEQ ID NO: 1); 1492R: 5′-GGTTACCTTGTTACGACTT-3′ (SEQ ID NO: 2)) were used for PCR; where a reaction system (30 μL) included: 6 μL of DNA template, 1.2 μL each of forward and reverse primers, 15 μL of Premix Taq™, and 6.6 μL of sterile enzyme-free water. The PCR program included: 94° C., 5 min; and 94° C., 30 S; 56° C., 30 S; 72° C., 45 S for 35 cycles in total; and 72° C., 10 min; the reaction was terminated at 4° C. and PCR products were temporarily stored. The PCR products were sent to Beijing RuiBiotech Co., Ltd. for sequencing. The sequencing results were aligned with the NCBI GenBank. The identification result was Parabacteroides distasonis, which was designated Parabacteroides distasonis F4.

### Example 3

Identification of bacterial physicochemical properties:

### I. Growth Curve

The above isolated and purified strain was activated in a solid medium and inoculated into a FAA liquid medium to allow overnight culture for 12 h. The obtained strain culture solution was re-inoculated into the liquid medium at a 2% (w/v) inoculation amount, mixed evenly and placed in a 37° C. anaerobic incubator for culture. The growth curve of the strain was determined by turbidimetry, where 200 μL of the bacterial solution was taken out in a certain interval, namely at 2 h, 4 h, 6 h, 8 h, . . . , until 22 h, 24 h, to measure absorbance at 600 nm. The result was shown in FIG. 3, indicating that the strain could grow stably.

### II. Resistance to Acid and Alkali

The FAB liquid medium was adjusted to pH values of 4, 5, 6, 7 and 8, respectively, and subjected to autoclaving for later use. The bacterial solution cultured overnight for 12 h was inoculated into each FAB liquid medium of each pH value at an inoculation amount of 10% (w/v), with the medium of pH=7 as a control. The culture was conducted in an anaerobic incubator at 37° C. for 24 h, and 200 μL of the bacterial solution from each tube was taken to determine its OD₆₀₀, and a survival rate of the strain at different pH values was calculated. The formula was: survival rate=(OD₆₀₀ of experimental group÷OD₆₀₀ of control group)×100%. The results shown in FIG. 4 indicate that the strain had certain resistance to acid and alkali.

### III. Resistance to Bile Salt

Liquid media with bile salt concentrations of 0%, 0.15%, 0.3%, and 0.6% were prepared, and subjected to autoclaving for later use. The bacterial solution cultured overnight for 12 h was inoculated into the FAB liquid medium with different bile salt concentrations at an inoculation amount of 10% (w/v), with the medium without bile salt as a control. The culture was conducted in a shaker at 37° C. for 10 h, and the OD₆₀₀ of each tube of bacterial solution was measured to calculate a survival rate of the strain under 3 bile salt concentrations. The formula was: survival rate=(OD₆₀₀ of experimental group÷OD₆₀₀ of control group)×100%. The results shown in FIG. 5 indicate that the strain had a certain survival rate in various bile salt concentrations.

### IV. Resistance to Artificial Gastric Fluid and Intestinal Fluid

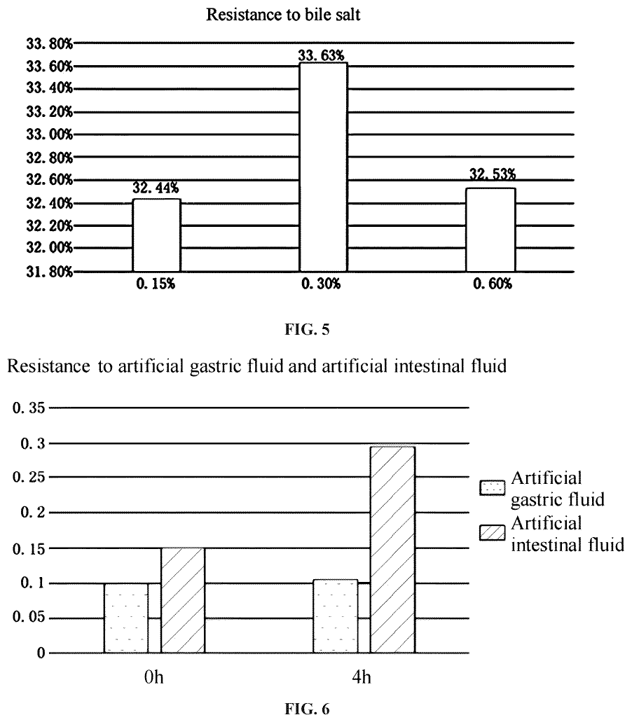

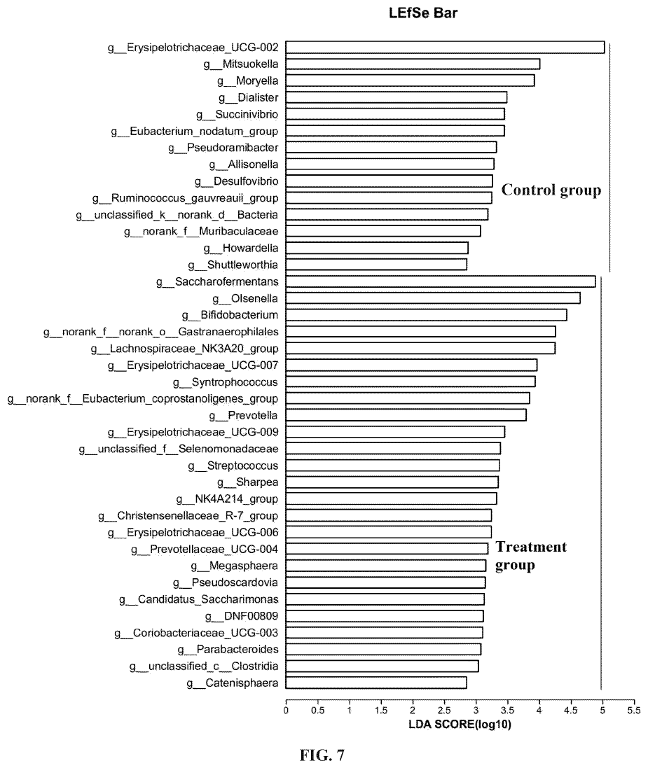

The overnight cultured bacterial solution was washed 2 times with PBS, and the bacterial cells were placed in artificial gastric fluid (FAB liquid medium with pH=3, autoclaved, cooled to room temperature, and into which 1 mg/mL pepsin was aseptically added in an ultra-clean bench) and artificial intestinal fluid (FAB liquid medium with pH=8, autoclaved, cooled to room temperature, and into which 1 mg/mL trypsin (1:250) was aseptically added in an ultra-clean bench). The bacterial solution was adjusted to a concentration of 10⁸ CFU/mL and then cultured in a 37° C. anaerobic incubator, and OD₆₀₀ of each bacterial solution was measured at 0 h and 4 h separately. The results shown in FIG. 6 indicate that the strain grew to a certain extent both in the artificial gastric fluid and artificial intestinal fluid and had high resistance.

### V. Acid Production Performance

The above purified strain was inoculated into FAB liquid medium and cultured anaerobically at 37° C. for 24 h. 0.5 mL of the bacterial solution was taken and added with 0.2 mL of ZnSO₄ and 0.1 mL of K₄Fe(CN)₆ solutions, shaken evenly, diluted with water to 5 mL, shaken evenly, centrifuged at 10,000 r/min for 10 min, 1 mL of a filtrate was taken after filtering through the membrane, diluted 5 times (to 5 mL) with 4 mL of water, and an acid production performance was detected by high-performance liquid chromatography (HPLC).

The results showed that the strain could produce lactic acid, acetic acid, propanoic acid, isobutyric acid, and isovaleric acid, with a propanoic acid yield of 981.75 μg/mL, a lactic acid yield of 669.50 μg/mL and an acetic acid yield of 228.70 μg/mL.

### Example 4

The example provided a microbial inoculant in the form of a fermentation broth or a freeze-dried powder, including the Parabacteroides distasonis F4 that has been deposited, where the fermentation broth contained 1×10⁹ CFU/mL of the live Parabacteroides distasonis F4, and the freeze-dried powder contained 1×10⁹ CFU/g of the live Parabacteroides distasonis F4.

### Example 5

This example provided a feed, including the microbial inoculant of Example 4, where the microbial inoculant was added in an amount of 1×10⁸ CFU/kg of the live Parabacteroides distasonis F4 into the feed.

### Example 6

In this example, an in vitro fermentation test was conducted on the Parabacteroides distasonis F4 and fermentation broth thereof:

Rumen fluid from adult fistulated Holstein cows was collected, filtered through four layers of gauze, and mixed with artificial saliva at a ratio of 1:2 to form artificial rumen fluid for in vitro fermentation test, in which a milk replacer was used as the substrate. Grouping of the test was shown in Table 1.

TABLE 1

| Grouping | Additives |
|---|---|
| Control group | FAB medium was added into the substrate |
| Metabolite group | Sterilized fermentation broth was added into the |
| | substrate |
| Parabacteroides | Parabacteroides distasonis F4 at 1 × 10⁸ CFU/mL |
| distasonis group | was added into the substrate |

A preparation method of fermentation rot in the Metabolite group included:
the Parabacteroides distasonis F4 in a logarithmic phase was inoculated into the fermentation medium (FAB) at an inoculation amount of 3%, then cultured for 48 h at a growth temperature of 37° C. and a growth pH value of 7.

The test results were shown in Table 2. The addition of Parabacteroides distasonis significantly reduced the ammonia nitrogen concentration (P<0.05), the addition of Parabacteroides distasonis significantly increased the proportions of propanoic acid content and butyric acid content in the rumen.

LefSe analysis shown in FIG. 7 indicated that compared with the control group, the group added with Parabacteroides distasonis (treatment group) significantly increased the abundance of potential beneficial bacteria such as Bifidobacterium spp., Christensenella spp., and Parabacteroides spp. in the animals, and improved the microbial balance in the rumen.

It was seen that the rumen-derived Parabacteroides distasonis F4 from calf screened in the present disclosure could effectively regulate the rumen microecological balance, change the rumen fermentation pattern, promote energy utilization and rumen development, and had great development potentials and application prospects in the future application of microbial inoculants for young ruminants.

TABLE 2

| | Control | Metabolite | Parabacteroides | | |
|---|---|---|---|---|---|
| Item | group | group | distasonis group | SEM | P-value |
| pH | 5.20 | 5.19 | 5.17 | 0.007 | 0.438 |
| Ammonia | 113.83^{a} | 113.78^{a} | 102.67^{b} | 2.084 | 0.009 |
| nitrogen | | | | | |
| (mg/dL) | | | | | |
| Microbial | 4.45 | 3.49 | 3.49 | 0.247 | 0.198 |
| protein | | | | | |
| (mg/dL) | | | | | |
| Acetic acid | 28.64 | 28.16 | 27.18 | 0.290 | 0.093 |
| (%) | | | | | |
| Propanoic | 18.91^{c} | 19.41^{b} | 20.38^{a} | 0.222 | <0.001 |
| acid (%) | | | | | |
| Butyric acid | 10.31^{ab} | 10.07^{b} | 10.59^{a} | 0.088 | 0.022 |
| (%) | | | | | |

NOTE:
different letters indicate statistical differences.

Finally, it should be noted that the foregoing embodiments are only used for illustration of the technical solutions of the present disclosure, and are not intended to limit the present disclosure. Although the present disclosure is described in detail with reference to the foregoing embodiments, those of ordinary skill in the art should understand that they can still modify the technical solutions described in the foregoing embodiments, or make equivalent substitutions to some technical features therein. These modifications or substitutions do not make the essence of the corresponding technical solutions depart from the spirit and scope of the technical solutions in the embodiments of the present disclosure.

What is claimed is:

1. A Parabacteroides distasonis strain, wherein the strain is Parabacteroides distasonis F4 with a deposit number of CGMCC No. 40798.

2. A microbial inoculant, comprising the Parabacteroides distasonis F4 according to claim 1 or a fermentation broth thereof.

3. The microbial inoculant according to claim 2, wherein the microbial inoculant is a freeze-dried powder of the fermentation broth of the Parabacteroides distasonis F4.

4. The microbial inoculant according to claim 3, wherein the freeze-dried powder of the fermentation broth further comprises a cryoprotectant; and the cryoprotectant is at least one selected from the group consisting of a skimmed milk powder, dextrin, lactose, and sucrose.

5. A product, comprising the Parabacteroides distasonis F4 according to claim 1; wherein the product is any one selected from the group consisting of a feed and a feed additive.

6. A product, comprising the microbial inoculant according to claim 2; wherein the product is any one selected from the group consisting of a feed and a feed additive.

7. The product according to claim 6, wherein the microbial inoculant is a freeze-dried powder of the fermentation broth of the Parabacteroides distasonis F4.

8. The product according to claim 7; wherein the freeze-dried powder of the fermentation broth further comprises a cryoprotectant; and the cryoprotectant is at least one selected from the group consisting of a skimmed milk powder, dextrin, lactose, and sucrose.

9. A method for preparing a product, comprising:
adding the Parabacteroides distasonis F4 according to claim 1 into a feed or a feed additive as the product.

10. A method for preparing a product, comprising:
adding the microbial inoculant according to claim 2 into a feed or a feed additive as the product.

11. The method according to claim 10, wherein the microbial inoculant is a freeze-dried powder of the fermentation broth of the Parabacteroides distasonis F4.

12. The method according to claim 11, wherein the freeze-dried powder of the fermentation broth further comprises a cryoprotectant; and the cryoprotectant is at least one selected from the group consisting of a skimmed milk powder, dextrin, lactose, and sucrose.

13. The method according to claim 9, wherein the product is used in increasing a content of propanoic acid in a rumen of a Holstein cow.

14. A method for increasing a content of propanoic acid in a rumen of a Holstein cow, comprising feeding the Parabacteroides distasonis F4 according to claim 1 to the Holstein cow.

15. A method for increasing a content of propanoic acid in a rumen of a Holstein cow, comprising feeding the microbial inoculant according to claim 2 to the Holstein cow.

16. The method according to claim 15, wherein the microbial inoculant is a freeze-dried powder of the fermentation broth of the Parabacteroides distasonis F4.

17. The method according to claim 16, wherein the freeze-dried powder of the fermentation broth further comprises a cryoprotectant; and the cryoprotectant is at least one selected from the group consisting of a skimmed milk powder, dextrin, lactose, and sucrose.

18. A method for increasing a content of propanoic acid in a rumen of a Holstein cow, comprising: adding the Parabacteroides distasonis F4 according to claim 1 or a fermentation broth thereof or a freeze-dried powder of the fermentation broth thereof into a feed, and
feeding the feed to the Holstein cow.

19. The method according to claim 18, wherein the Parabacteroides distasonis F4 is added in an amount of more than 1×10⁸ colony-forming unit (CFU) per kg of the feed.
